(19) United States
(12) Patent Application Publication
(10) Pub. No.: US 2007/0088261 A1
(43) Pub. Date: Apr. 19, 2007

(54) Hypodermic needle safety cap apparatus

(76) Inventors: Jung Chul Lew, Windsor Locks, CT (US); Sean Won Lew, (US); Christopher S. Yi, (US); Marian M. Yi, (US)

Correspondence Address: Dr. Jung Chul Lew, 28 Dibble Hollow Lane, Windsor Locks, CT, 06096, US

(21) Appl. No.: 11/507,826

(22) Filed: Aug. 22, 2006

Related U.S. Application Data
(60) Provisional application No. 60/723,088, filed on Oct. 3, 2005.

(51) Int. Cl. A61M 5/00

(52) U.S. Cl. 604/110; 604/111

(57) ABSTRACT

FIG. 1 depicts two different size open slots; the narrow opening guides the needle by its slot during Step 2 procedure (FIG. 4). The lager open slot (40) provided at the end of the needle cap (FIG. 1) is the clearance for the needle hub (100 in FIG. 4). This open slot (40) allows the needle to come in at angle to make a contact with the cap at 90 (FIG. 4). This procedure eliminates the conventional way of capping of pushing against each other between a cap and a needle where the most percutaneous injuries are occurred while doing so.

When it reaches the position Step 3 in FIG. 4, the tip of the needle is already passed the injury plane which means the hand and fingers are behind the point where it is safe to push the cap against the needle away from the hand of the user.

### CROSS-REFERENCE TO RELATED APPLICATIONS

This application claims the benefit of U.S. Provisional Patent Application No. 60/702,396 (Confirmation No.: 9879), filed Jul. 27, 2005 and 60/723,088 (Confirmation No.: 9725), filed Oct. 03, 2005 by the present inventors.

### FEDERALLY SPONSORED RESEARCH

Not Applicable

### SEQUENCE LISTING OR PROGRAM

Not Applicable

### BACKGROUND OF THE INVENTION

1. Field of Invention

This invention relates to hypodermic syringe apparatus for needle safety, specifically to a device to protect from injury due to accidental needle sticks eliminating the risk of accidental needle-sticks and particularly, to these instruments for use in fine needle biopsy of human for medical diagnostics and many other intravenous equipment and promoting human health by decreasing prevalence of serious infectious diseases caused by percutaneous injuries from the accidental needle sticks such as Human Immunodeficiency Virus (HIV) and Hepatitis.

2. Background of the Invention

Needle stick injuries are one of the most common job related injuries among health care workers. There are an estimated one million percutaneous injuries (accidentally getting stuck with an exposed needle during a medical procedure) among health care workers occur annually a year. This costs the medical industry billions of dollars annually to test and treat injured health care workers.

Numerous studies show that conventional two-handed recapping is the cause of a significant portion of all needle-stick injuries. Nurses are the most likely group to have received exposure and that the vast majority of the exposures were the result of needle-stick injuries. Otherwise, the uncapped needles are thrown into permanent sealable containers (e.g., sharps containers). Also, while disposing of uncapped needles into sealable containers sounds like an ideal solution to the needle-stick problem, in actual practice, not all needles are properly disposed of, and needles stuffed into overfilled containers may still be dangerous. Again, the above strengthens the need for safety needle caps. This invention of the safety needle recapping device can contribute to the highest safe operation of the syringe in the health care work environment at a fraction of a cost.

Our invention will eliminate the chance of getting stuck by exposed needles while capping and will concurrently save a substantial amount of money in health care. This patented instrument meets all directives relating to safety devices to prevent needle sticks published by the National Institute for Occupational Safety and Health. As shown in FIG. 1 through 4, this patented device requires no activation by the user. The safety feature is integral part of the instrument with no added components. It is quite evident that this invented feature is safe and effective for patient care and at the same time performs reliably and remains protective through disposal for health care workers.

Since the invention of the hypodermic needle, inventors created numerous types of safety needle caps in such a way as to protect the user during and after the procedure. For example, U.S. Pat. No. 4,139,009, U.S. Pat. No. 5,630,803, U.S. Pat. No. 5,964,731, U.S. Pat. No. 5,356,387, U.S. Pat. No. 5,411,492, U.S. Pat. No. 6,183,445 B1, U.S. Pat. No. 6,093,170, U.S. Pat. No. 4,113,090, U.S. Pat. No. 5,415,645, U.S. Pat. No. 5,069,669, U.S. Pat. No. 4,955,866, U.S. Pat. No. 5,256,152, U.S. Pat. No. 5,538,508, U.S. Pat. No. 5,295,972, U.S. Pat. No. 5,250,031, U.S. Pat. No. 4,659,330 and more have been developed and marketed but found ineffective in protecting the users and in many cases, it makes it more dangerous to the users and patients. Many aforementioned inventions obstruct view during the operation of the device. For example, U.S. Pat. No. 4,867,172, U.S. Pat. No. 6,015,397, U.S. Pat. No. 5,700,249, U.S. Pat. No. 3,840,008 discloses a complex sliding mechanism which can only protect partially; however these sliding mechanisms are prohibitively expensive to manufacture and a large area of the used needle axis is exposed when the needle safety device is in place to guard the needle point. In such condition the partially uncovered device constitutes a serious hazard indirectly which can affect disease transfer to the individual making contact with same. Also, these devices are not utilizing passive activation concept in the design which is not user friendly and unreliable. In order to find a right spot on patient's skin for a medical injection to or to withdraw blood from a patient, the needle safety cap must be pushed back to see and guide the needle point for the procedure.

There are a few passive needle safety devices which enhance the safety design and are more likely to have a greater impact on prevention than active devices such as U.S. Pat. No. 5,295,972, U.S. Pat. No. 5,147,303, U.S. Pat. No. 5,630,803, U.S. Pat. No. 4,139,009 and U.S. Pat. No. 6,319,233 B1; however, these devices are very complex with many parts, and most cannot be used with standard injection devices. However, these are generally large in size and have suffered from the disadvantages of being relatively expensive in a field where lower cost is of the greatest priority due to the large volume required.

Our own patent 60/723,088 PPA confirmation No. 9725 describes a needle safety cap which is made of flexible disposable plastic which requires a low cost to manufacture while also providing maximum protection. Nevertheless all other needle safety cap devices heretofore known suffer from a number of disadvantages:
(a) Too expensive to manufacture.
(b) Most devices dose not fully provides protection.
(c) Most devices are not designed to be disposable.
(d) Obstruct view during medical procedure.
(e) Consists too many complex parts to provide protection.
(f) Most devices are not fully implemented passive design.
(g) Most devices are not simple to use.
(h) Most devices are non-compatible.
(i) Most devices are not always practical.

### BACKGROUND OF THE INVENTION—OBJECTS AND ADVANTAGES

It is therefore an object of the present invention to solve the problems discussed above relating to the existing design of the needle caps and to provide a safer way of both capping and disposal of the needle after being used and to provide a novel inexpensive and effective protective device of the kind described at a fraction of the current cost of manufacturing.

Furthermore it is an object of this invention to provide an improved needle safety cap device for protecting users to prevent accidental needle sticks and the accompanying risk of transmission of blood-born pathogens such as HIV and Hepatitis.

It is also an object of the current invention to allow for universal adaptability to different sizes and types of hypodermic syringes, blood collection needles sets, intravenous catheters, and other intravenous infusion equipment by providing disposable and interchangeable safety needle cap device.

Further objects and advantages of our invention will become apparent from a consideration of the drawings and ensuing description.

Accordingly, several objects and advantages of the present invention are:

(a) to provide a needle safety cap device that uses an improved simple design that can prevent accidental needle sticks effectively.
(b) to provide a needle safety cap such that the device works passively and requires no activation by the user to perform.
(c) to provide a needle safety cap device that uses an existing design but improved without adding additional parts to enhance the performance but to provide more reliable protection.
(d) to provide a needle safety cap device that does not modify the syringe.
(e) to provide a needle safety cap device which is compatible with all needle injection devices.
(f) to provide a needle safety cap device that remains protective through disposal.
(g) to lower manufacturing cost but improve functionality.

The invention differs from known devices for the same purpose in a number of ways. It is a passive system, unlike most other needle safety devices. Furthermore, this invention modifies the cap without adding any additional parts in order to provide protection than the current design, and can be fitted on the full range of medical equipment using needles. Other devices for the same purpose modify the entire piece of equipment, for example the entire hypodermic syringe or the entire catheter. Our device is easy to manufacture. It is made of plastic part and there are no parts to assemble because our invented safety feature is incorporated as one piece into a needle cap. The invention is designed to fit the standard needle hub which will attach to the standard syringe, eliminating the need for a different needle or syringe system.

### SUMMARY

In accordance with the present inventions, three different types of needle safety cap devices have been invented to reduce the risk of accidental needle sticks and for safe disposal. One embodiment of the present invention relates to a disposable needle safety cap with a built-in open slot. The second embodiment of the present invention relates to a disposable needle safety cap with a built-in spiral needle guide opening. Another embodiment of the present invention relates to a disposable safety needle cap with a built in automatic self enclosure within the needle safety cap device as illustrated in FIG. #1, 2 and 3.

### DRAWINGS—FIGURES

The invention is diagrammatically illustrated, by way of example, in the drawings appended hereto in which:

FIG. 1 is front, top and section views of the needle safety cap device illustrating the location of the slot within the device according to the present invention. This device is a side-entry-at-angle design;

FIG. 2 is front, top and section views of the needle safety cap device illustrating the location of the slot within the device according to the present invention. This device is a-side-entry-at-angle design with an automatic self closing feature built in within the entry slot;

FIG. 3 is front, top and section views of the needle safety cap device illustrating the location of the slot within the device according to the present invention. This device is a side-entry-at-angle to radial entry design.

FIG. 4 is a view of the needle capping sequence using the invented safety cap illustrating how the needle is capped within the device. This figure is shown with a FIG. 1 Safety cap device (Side entry at angle design).

### DRAWINGS—LIST OF REFERENCE NUMERALS

10. Safety cap body—Model #1
20. Safety holding tab
30. Needle guiding slot—Color coated in green
40. Opening where needle hub (110) is inserted at angle
50. Safety cap body—Model #2
60. Automatic self enclosure—Double fins
70. Safety cap body—Model #3
80. Spiral opening where needle is inserted
90. Pivoting point where needle hub is making first contact
100. Needle hub
110. Tapered needle hub
120. Needle locked in secured position
130. Cap cavity for needle
140. Automatic self enclosure—Single fin.
150. Label—“Safety Hold”
160. Integrated Needle Holder

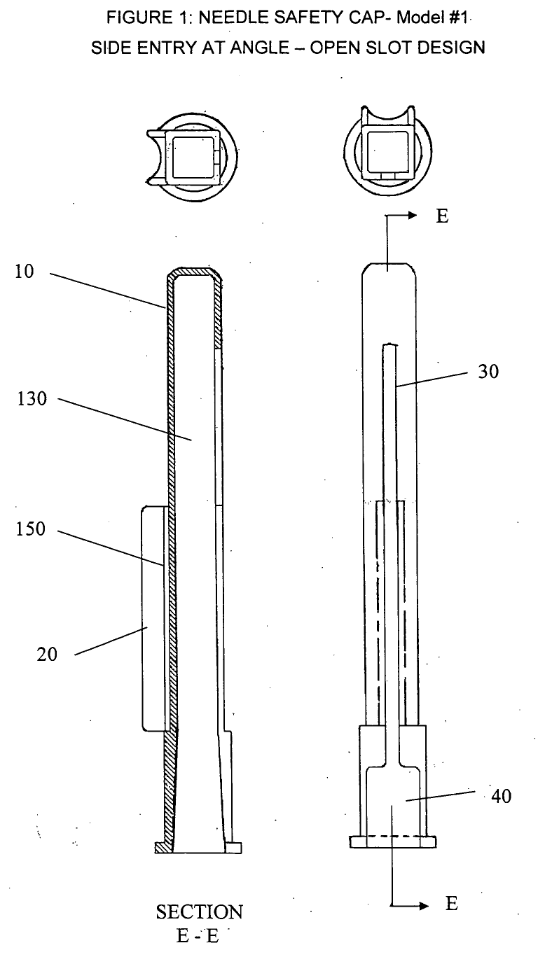

### DETAILED DESCRIPTION—FIGS. 1 THROUGH 4—PREFERRED EMBODIMENT

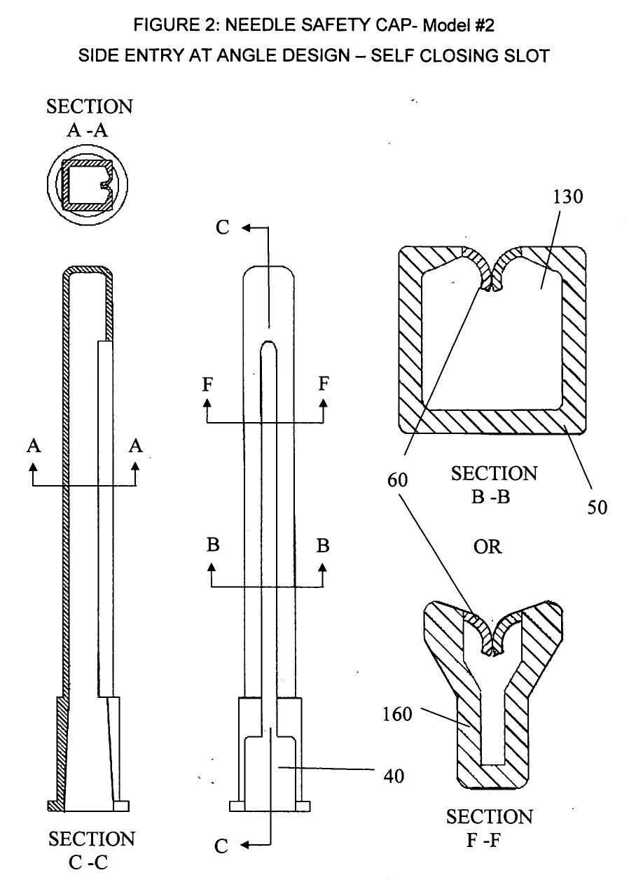

In accordance with the present inventions, there are four different types of needle safety cap devices; FIG. #1 comprises an open slot to the most commonly known needle caps. FIG. #2 comprises both the open slot safety cap and a self closing device incorporated (Item 60) into the cap; FIG. #3 comprises the spiral safety cap device within the cap with a self closing device incorporated (140) and lastly FIG. 2, Section F-F comprises a space saving open slot with a self closing device incorporated (160) which have been developed for better control of the safety needle cap to reduce the risk of accidental needle sticks.

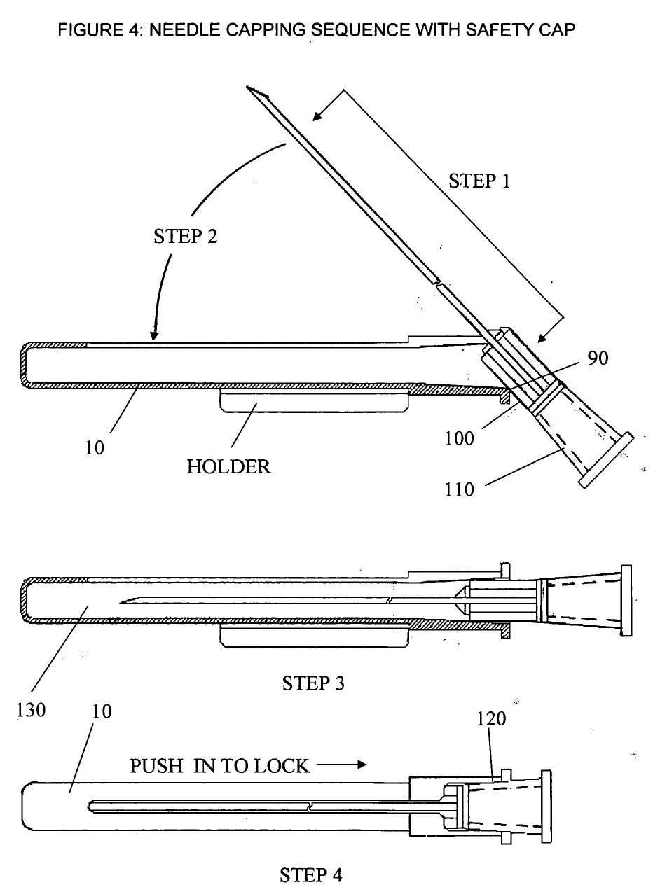

As shown in FIG. 1, the present invention shows front, top and sectional views of the before mentioned needle safety cap. In the preferred embodiment, the safety cap is a plastic material comprises two openings: first one is an opening 40 for a needle hub 100 to be engaged in with a needle cap at position 90, the second one is an elongated open slot 40 for guiding the needle to travel through as the needle assembly is pivoted at 90 as illustrated in FIG. 4 how the standard needle hub 100 is inserted, and shows point 40 is where the needle hub 100 enters into the invented safety needle making a contact with point 10. Position 20 illustrates a safety holding tab integrated into point 10 as part of one molded piece which is used both for assembly of the needle safety cap device and the function of preventing it from rolling as a mechanism within the needle safety cap device. Also, point 20 within the invention of the safety cap assists the user in identifying where to hold within the safety cap for safe usage. In the preferred embodiment, the “Safety Hold” label 150 is placed on 20. The width of the slot is dependent of the gauge of the needle in order to minimize the opening, therefore it minimize the chances of any unwanted blood-born pathogens to leak.

Additional embodiment is shown in FIG. 2. In this case, open slot 30 is replaced with 60 replacing the opening 30 with very flexible thin plastic or thin rubber on the external edge surface of the slotted surface on 30 from both ends extending towards the center slightly pressing on against each other forming an arc as a result as illustrated in FIG. 2, while fins 60 is incorporated into the internal surface of needle slot 30. As a result, it is closed at all time. The same system of operation is employed as FIG. 1. The advantage of this embodiment of 60 is that it allows any needle gage size to pass through and contains any unwanted blood-born pathogens by an automatic self closing feature 60.

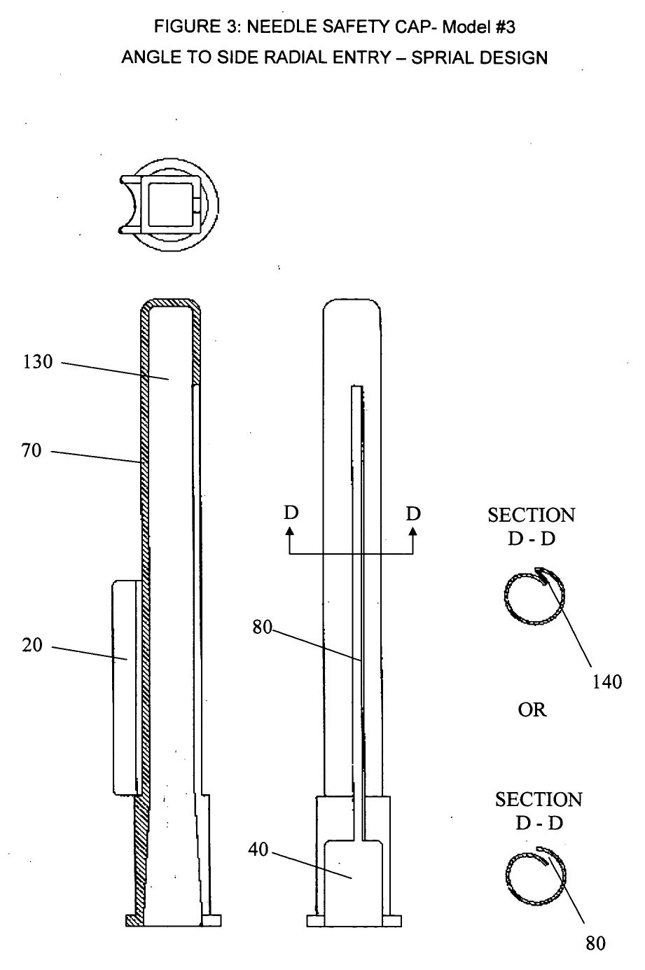

Additional embodiment is shown in FIG. 3. In this case, open slot 30 is replaced with the spiral shape body of the safety cap 80 and a single fin 140 replacing the opening 30 with very flexible thin plastic or thin rubber on the internal edge surface of the spiral safety cap while fins 140 are incorporated into the internal surface of 70. As a result, this needle cap with syringe assembly can be placed on the table at any orientation and still be able to provide without any potential leakage from it which can cause health risk.

### OPERATION—FIGURE 4: SAFETY CAP DEVICE CAPPING SEQUENCE

What is shown on page FIG. 4 is the capping sequence with a 1-½″ needle and its operations how to use the patented needle safety cap. The length of the slot in the cap will be dependent on the length of the needle accordingly.

As shown in FIG. 4, place the needle at the pivot area (90 in FIG. 4) in the cap at about a 45 degree angle having the plastic body portion (100) of the needle inside the cap where the open slot (40) is provided. After step 1, simply pivot around the point (90) reducing the angle to zero. During this procedure, the needle shaft will follow the elongated open slot, guiding the tip of the needle safely capturing the needle stick into the cap cavity. It does not matter how long the needle is because whatever the length it might be, the needle will follow the slot provided. When the needle is captured within the cap cavity, simply push the cap to snap it on. At that point, it is safe to apply a counter force to lock it in because the tip of the needle is inside the cap and away from the fingers.

At this time, the whole needle safety cap device with the syringe can be disposed of without the possibility of accidental needle stick injury.

By far, this simple procedure using the patented needle safety cap as mentioned above can significantly reduce the number of accidental needle stick injuries for needles used in clinical situations, for example, attached to hypodermic syringes.

This needle safety cap device covers the needle both before and after the injection for safety.

### CONCLUSION, RAMIFICATIONS, AND SCOPE OF INVENTION

Accordingly, the reader will see that the needle safety device of the invention provides a passive mechanism of action that can easily be attached to the hub of any standard needle without modifying the syringe system. The pivotal point of this invention is the way the needle's sharp point approaches the cap. It is important to remember that the reason behind many of the needle related injuries is primarily because conventional needle caps have such a minute opening and the way the needle is being inserted into the cap longitudinally. The cap's target of less than a quarter inch in diameter must be hit with impeccable accuracy if injury is to be avoided. This makes the probability of injury very high and it has been proven. However, if the axis of the needle turned about 30 to 45 degrees to the longitudinal axis of the cap, the needle point will be facing away from the hand of the user as opposed to directly at it as illustrated in FIG. 4. This simple one step eliminates the chance for injuries. Our invented needle safety cap apparatus provides a way of capping without ever pointing the sharp needle point to operator's hand directly in order to cap it.

The health care professionals can use the same standard syringe with the addition of this safety cap without altering their technique. There is no difference in technique uncapping. The device can be used easily by the health care professional by bringing the needle at angle as illustrated in FIG. 4. There are many favorable advantages of the invention of the needle safety cap device; its simplicity, user friendliness, lower manufacturing cost and effectiveness in preventing percutaneous injury. While our above description contains many specificities, these should not be construed as limitations on the scope of the invention, but rather as an exemplification of one preferred embodiment thereof. Many other variations are possible. For example, there can be any number of disposable caps if needed to cover more length of the needle meaning the needle entry slot can be shorter or longer depending on the length of the needle. Also, the shape of the disposable safety cap can differ from the cylindrical shape, for example the shields can be spiral shape for the needle stick entry. The most important ramification is the clearance provided on the outer surface of the needle safety cap allowing the needle stick to be entered from the side of the invented needle safety cap.

Accordingly, the scope of the invention should be determined not by the embodiments illustrated, but by the appended claims and their legal equivalents.

We claim:

1. A safety hypodermic needle cap apparatus adapted to be coupled to a standard needle hub assembled with syringe provided at one end with a projecting nozzle shape cap surrounded by a coupling collar; said assembly comprising:
A. a hollow housing receivable in the hub of the needle and having a housing mounted therein provided with a tapered points securing the housing in the needle hub.
B. a said housing having an entrance opening, wherein said needle housing is capable of being entered at angle or removed from said housing at angle without opening said housing.
C. a slot longitudinally elongated in the center to said housing; said opening of said slot is passively operative for guiding a hypodermic needle in order to capture the needle tip inside said housing cavity by turning said needle to zero degree.
D. a color coated in green to said elongated slot surface is for identifying the location where to engage with ease for safer capping.
E. a safety tab incorporated into said housing is to prevent from rolling and to orient the housing automatically for insertion of the needle.

2. The passive hypodermic needle safety cap apparatus of claim 1, wherein said safety cap is disposable following use on a single patient.

3. The safety needle cap of claim 1 wherein the cap, the slot longitudinally elongated and the entrance opening are one piece integral synthetic clear thermoplastic material such as polycarbonate.

4. The hypodermic needle cap apparatus of claim 1 wherein said body is capable of being self closed automatically by having two self needle closing enclosures which is molded as one piece integral with the cap.
A. a thin and flexible synthetic thermoplastic material is molded on both sides of the elongated slot surfaces to form a closure to claim 1.

5. As an alternative to claim #1 aforementioned, the shape of the said cap body is spiral with a self closing enclosure molded as one piece integral with the cap.
A. the hypodermic needle cap apparatus of claim 5 wherein said body is spiral member comprising a thermoplastic material which is relatively stiff member.
B. the hypodermic needle cap apparatus of claim 5 wherein said body is capable of being self closed automatically by having one self needle closing enclosure which is molded as one piece integral with the cap.

6. As an alternative to claim #1 aforementioned, the contour of the said cap body is “Y” shaped capturing the needle into the cavity with a self closing enclosure molded as one piece integral with the cap.
